(19) United States
(12) Patent Application Publication
(10) Pub. No.: US 2026/0308035 A1
(43) Pub. Date: Oct. 8, 2026

(54) Method for Treating Phosphorus-Containing Wastewater Produced During Preparation of Sulfonamide Compound

(71) Applicant: Nanhai Beisha Pharmaceutical Co., Ltd., Foshan (CN)

(72) Inventors: Sheng HUANG, Foshan (CN); Weijie QIU, Foshan (CN); Yiming YUAN, Foshan (CN)

(21) Appl. No.: 19/441,624

(22) Filed: Jan. 6, 2026

(30) Foreign Application Priority Data
Jan. 22, 2025 (CN) 202510104296.X

(51) Int. Cl. C02F 9/00; C07D 237/20; C07D 241/44; C02F 1/28; C02F 1/52; C02F 101/10; C02F 101/30; C02F 103/36

(52) U.S. Cl. CPC C02F 9/00; C07D 237/20; C07D 241/44; C02F 1/283; C02F 1/5209; C02F 2001/5218; C02F 1/5245; C02F 2101/105; C02F 2101/308; C02F 2103/36; C02F 2301/08

(57) ABSTRACT

Provided is a method for treating phosphorus-containing wastewater produced during preparation of a sulfonamide compound. The method includes: adjusting a pH of a condensed aqueous phase to 5.5 to 6.0 and conducting a first solid-liquid separation to obtain a first solid component and a first mother liquor, respectively; adjusting a pH of the first mother liquor to 12 to 13 by using sodium hydroxide, cooling a resulting material to 0°C to 10°C, and conducting crystallization and a second solid-liquid separation in sequence to obtain a second solid component and a second mother liquor, respectively; and mixing the second mother liquor and calcium chloride and conducting a precipitation reaction, and subjecting a resulting reaction material to a third solid-liquid separation to obtain a third solid component and a final mother liquor, respectively; and drying the third solid component to obtain calcium phosphate.

### CROSS REFERENCE TO RELATED APPLICATION

This patent application claims the benefit and priority of Chinese Patent Application No. 202510104296.X filed with the China National Intellectual Property Administration on Jan. 22, 2025, the disclosure of which is incorporated by reference herein in its entirety as part of the present application.

### TECHNICAL FIELD

The present disclosure relates to the technical field of pharmaceutical technology, and in particular to a method for treating phosphorus-containing wastewater produced during preparation of a sulfonamide compound.

### BACKGROUND

Sulfonamide drugs are the first artificially synthesized antibacterial drugs, typically appearing as white or pale yellow crystalline powders. Since invention and application in the 1930s, sulfonamide drugs have been highly valued and extensively researched as antibacterial drugs with the most research and application value due to advantages such as easy production, easy storage, good efficacy, convenient administration, and low price. Over the past 80 years, more than 8500 types of sulfonamide drugs have been synthesized, of which over 20 are in common clinical use, such as sulfadiazine, sulfamonomethoxine, and sulfadimidine. With the continuous discovery and development of various antibiotics, sulfonamide drugs have been gradually replaced by antibiotics and quinolones. Nevertheless, sulfonamide drugs still have unique advantages, such as a relatively broad antibacterial spectrum, stable properties, easy administration, low cost, absence of grain consumption during production, and the capacity for large-scale manufacturing. As a result, sulfonamide drugs remain one of the important drugs for anti-infective treatment in livestock and poultry.

However, the practical production of sulfonamide drugs generates a variety of wastewater streams, which are complex to treat. Among the wastewater streams, phosphorus-containing wastewater, often treated via distillation, yields final inorganic salts that cannot be recycled, alongside high-cost solid waste treatment.

### SUMMARY

In view of this, the present disclosure is to provide a method for treating phosphorus-containing wastewater produced during preparation of a sulfonamide compound. In the present disclosure, the method yields trisodium phosphate and calcium phosphate from the phosphorus-containing wastewater produced during the preparation of the sulfonamide compound, resulting in a final mother liquor with a low phosphorus content.

In order to achieve the above object, the present disclosure provides the following technical solutions:

The present disclosure provides a method for treating phosphorus-containing wastewater produced during preparation of a sulfonamide compound, including the following steps:

removing a solvent in a crude product solution of the sulfonamide compound and then adding water, or adding the water to the crude product solution of the sulfonamide compound and layering, to obtain a condensed aqueous phase, and adjusting a pH of the condensed aqueous phase to 5.5 to 6.0, and conducting solid-liquid separation to obtain a first solid component and a first mother liquor, respectively, and drying the first solid component to obtain the sulfonamide compound, where the crude product solution of the sulfonamide compound is obtained by subjecting phosphorus oxychloride and a hydroxyl compound to a chlorination reaction, and subjecting a resulting reaction product to acidification at a pH of 3 to 3.5, and subjecting a resulting acidification product to a condensation reaction with sulfonamide; and an acid for adjusting the pH to 5.5 to 6.0 includes an acid obtained by the acidification;

adjusting a pH of the first mother liquor to 12 to 13 by using sodium hydroxide, cooling a resulting material to 0°C to 10°C, and conducting crystallization and solid-liquid separation in sequence to obtain a second solid component and a second mother liquor, respectively; and drying the second solid component to obtain trisodium phosphate; and

mixing the second mother liquor and calcium chloride and conducting a precipitation reaction, and subjecting a resulting reaction material to solid-liquid separation to obtain a third solid component and a final mother liquor, respectively; and drying the third solid component to obtain calcium phosphate.

In some embodiments, the method further includes:

before adding the water to the crude product solution of the sulfonamide compound, removing the solvent in the crude product solution of the sulfonamide compound;

decolorizing the condensed aqueous phase by using a first activated carbon before adjusting the pH of 5.5 to 6.0; and

decolorizing the first mother liquor by using a second activated carbon and then conducting solid-liquid separation before adjusting the pH of 12 to 13 by using sodium hydroxide.

In some embodiments, the method further includes, before cooling to 0°C to 10°C, decolorizing the resulting material by using a third activated carbon; and

the crystallization is conducted for 1 h to 2 h.

In some embodiments, a molar ratio of the calcium chloride to trisodium phosphate in the second mother liquor is in a range of 1.5-1.6 : 1.

In some embodiments, the crude product solution of the sulfonamide compound is prepared by a process including the following steps:

subjecting the phosphorus oxychloride and the hydroxyl compound to the chlorination reaction to obtain a chlorination reaction solution;

mixing the chlorination reaction solution obtained after the chlorination reaction, a water-immiscible solvent, and ice water, and adjusting a pH of a resulting mixture to 3 to 3.5, and layering to obtain an organic phase and an acidic aqueous phase, respectively; evaporating the organic phase to dryness to obtain a chloride; and recycling the acidic aqueous phase back to a step of adjusting the pH to 5.5 to 6.0; and

mixing the chloride, sulfonamide, an organic solvent, and an alkaline reagent, and conducting the condensation reaction to obtain the crude product solution of the sulfonamide compound.

In some embodiments, the hydroxyl compound is selected from a group consisting of maleic hydrazide and 2-hydroxyquinoxaline;

a molar ratio of the hydroxyl compound to the phosphorus oxychloride is in a range of 1 : 2-2.2; and

the chlorination reaction is conducted at a temperature of 100°C to 105°C for 1.5 h to 2 h.

In some embodiments, the water-immiscible solvent includes at least one selected from the group consisting of dichloromethane, chloroform, dichloroethane, and toluene; and

an alkali for adjusting the pH of the resulting mixture to 3 to 3.5 includes at least one selected from the group consisting of sodium hydroxide, sodium carbonate, and sodium bicarbonate.

In some embodiments, a molar ratio of the sulfonamide to the chloride is in a range of 1 : 0.95-1;

the alkaline reagent includes at least one selected from the group consisting of sodium carbonate and sodium bicarbonate, and a molar ratio of the sulfonamide to the alkaline reagent is in a range of 1: 1-1.45; and

the organic solvent includes at least one selected from the group consisting of o-dichlorobenzene and dimethylformamide.

In some embodiments, the condensation reaction is conducted at a temperature of 145°C to 170°C for 3.5 h to 4 h.

In some embodiments, the process further includes decolorizing the acidic aqueous phase by using a fourth activated carbon before use.

In the present disclosure, phosphorus oxychloride and a hydroxyl compound undergo a chlorination reaction, followed by acidification at a pH of 3 to 3.5 to produce a chloride, hydrochloric acid, and phosphoric acid, and then a condensation reaction with sulfonamide to produce a sulfonamide compound and sodium carbonate. In the present disclosure, by means of neutralization between the hydrochloric acid and the phosphoric acid (acidic aqueous phases) produced during the preparation of the sulfonamide compound and the sodium carbonate produced during the condensation reaction, acid and alkali consumption in the preparation of the sulfonamide compound and in the treatment of phosphorus-containing wastewater is reduced; the pH of a condensed aqueous phase is adjusted to 5.5 to 6.0 to produce sodium dihydrogen phosphate and disodium hydrogen phosphate, and sodium hydroxide is then added to adjust the pH to 12 to 13 to completely convert the sodium dihydrogen phosphate and the disodium hydrogen phosphate into trisodium phosphate. The trisodium phosphate is separated by utilizing the significant difference in solubility between the trisodium phosphate and sodium chloride at a low temperature of 0°C to 10°C. Finally, calcium chloride and the trisodium phosphate are reacted to form a calcium phosphate precipitate, and thus phosphate ions are substantially removed from a second mother liquor via solid-liquid separation, and sodium chloride remains in a final mother liquor, thereby achieving the full recovery of inorganic salts from the phosphorus-containing wastewater and the goal of advanced phosphorus removal from the phosphorus-containing wastewater. The present disclosure provides a novel idea for treating the phosphorus-containing wastewater and considers the treatment of the phosphorus-containing wastewater in the process design stage, thereby offering a new alternative for treating the phosphorus-containing wastewater from other similar products. Furthermore, in the present disclosure, the method can reduce the production costs of enterprises, and in particular can reduce the cost of solid waste treatment.

Further, in the present disclosure, a water-immiscible solvent and ice water are used as a hydrolysis system for phosphorus oxychloride and a pH is adjusted to 3 to 3.5, thereby essentially neutralizing the hydrochloric acid in a phosphorus oxychloride hydrolysate into sodium chloride while retaining the phosphoric acid. Furthermore, such pH range allows for the complete separation of chlorides from water by extraction using the water-immiscible solvent.

### DETAILED DESCRIPTION OF THE EMBODIMENTS

The present disclosure provides a method for treating phosphorus-containing wastewater produced during preparation of a sulfonamide compound, including the following steps:

removing a solvent in a crude product solution of a sulfonamide compound and then adding water, or adding the water to the crude product solution of the sulfonamide compound and layering, to obtain a condensed aqueous phase, and adjusting a pH of the condensed aqueous phase to 5.5 to 6.0 and conducting solid-liquid separation to obtain a first solid component and a first mother liquor, respectively, and drying the first solid component to obtain the sulfonamide compound, where the crude product solution of the sulfonamide compound is obtained by subjecting phosphorus oxychloride and a hydroxyl compound to a chlorination reaction, and subjecting a resulting reaction product to acidification at a pH of 3 to 3.5, and subjecting a resulting acidification product to a condensation reaction with sulfonamide; and an acid for adjusting the pH to 5.5 to 6.0 includes an acid obtained by the acidification;

adjusting a pH of the first mother liquor to 12 to 13 by using sodium hydroxide,

and cooling a resulting material to 0°C to 10°C, and conducting crystallization and solid-liquid separation in sequence to obtain a second solid component and a second mother liquor, respectively; and drying the second solid component to obtain trisodium phosphate dodecahydrate;

mixing the second mother liquor and calcium chloride and conducting a precipitation reaction, and subjecting a resulting reaction material to solid-liquid separation to obtain a third solid component and a final mother liquor, respectively; and drying the third solid component to obtain calcium phosphate.

Unless otherwise specified, the materials and equipment used in the present disclosure are commercially available in the art.

The preparation of the crude sulfonamide compound is described in detail below.

In some embodiments of the present disclosure, the crude product solution of the sulfonamide compound is prepared by a process including the following steps:

subjecting the phosphorus oxychloride and the hydroxyl compound to the chlorination reaction to obtain a chlorination reaction solution;

mixing the chlorination reaction solution obtained after the chlorination reaction, a water-immiscible solvent, and ice water, and adjusting a pH of a resulting mixture to 3 to 3.5, and layering to obtain an organic phase and an acidic aqueous phase, respectively; evaporating the organic phase to dryness to obtain a chloride; and recycling the acidic aqueous phase back to a step of adjusting the pH to 5.5 to 6.0; and

mixing the chloride, sulfonamide, an organic solvent, and an alkaline reagent and conducting the condensation reaction to obtain the crude product solution of the sulfonamide compound.

In some embodiments of the present disclosure, the phosphorus oxychloride and the hydroxyl compound are subjected to the chlorination reaction to obtain the chlorination reaction solution.

In some embodiments of the present disclosure, the hydroxyl compound is selected from a group consisting of maleic hydrazide and 2-hydroxyquinoxaline. In some embodiments of the present disclosure, a molar ratio of the hydroxyl compound to the phosphorus oxychloride is in a range of 1 : 2-2.2, and is 1 : 2, 1 : 2.05, 1 : 2.1, 1 : 2.15, or 1 : 2.2 in a particular embodiment.

In some embodiments of the present disclosure, the chlorination reaction is conducted at a temperature of 100°C to 105°C, and is 100°C, 101°C, 102°C, 103°C, 104°C, or 105°C in a particular embodiment; and the chlorination reaction is conducted for 1.5 h to 2 h, and is 90 min, 100 min, 110 min or 120 min in a particular embodiment. In the present disclosure, the chlorination reaction solution is used directly in the subsequent reaction without any workup.

In some embodiments of the present disclosure, after the chlorination reaction solution is obtained, the chlorination reaction solution obtained after the chlorination reaction, the water-immiscible solvent, and the ice water are mixed, and the pH of the resulting mixture is adjusted to 3 to 3.5 and a resulting system is subjected to layering to obtain the organic phase and the acidic aqueous phase, respectively; the organic phase is evaporated to dryness to obtain the chloride; and the acidic aqueous phase is recycled back to the step of adjusting the pH to 5.5 to 6.0.

In some embodiments of the present disclosure, the water-immiscible solvent includes at least one selected from the group consisting of dichloromethane, chloroform, dichloroethane, and toluene. In some embodiments of the present disclosure, a ratio of a mass of the hydroxyl compound to a volume of the water-immiscible solvent is in a range of 1 g: 4-6 mL, and is 1 g : 4 mL, 1 g : 4.5 mL, 1 g : 5 mL, 1 g : 5.5 mL, or 1 g : 6 mL in a particular embodiment. In some embodiments of the present disclosure, a volume ratio of the first organic solvent to the ice water is in a range of 1 : 0.55-0.65, and is 1 : 0.55, 1 : 0.6 or 1 : 0.65 in a particular embodiment.

In some embodiments of the present disclosure, mixing the chlorination reaction solution obtained after the chlorination reaction, the water-immiscible solvent, and the ice water is conducted by: adding the chlorination reaction solution dropwise to the water-immiscible solvent and the ice water, where during dropwise addition, a temperature of a system is in a range of 15°C to 25°C, and is 15°C, 20°C or 25°C in a particular embodiment.

In some embodiments of the present disclosure, an alkali for adjusting the pH to 3 to 3.5 includes at least one selected from the group consisting of sodium hydroxide, sodium carbonate, and sodium bicarbonate. In a particular embodiment, the pH is 3, 3.1, 3.2, 3.3, 3.4 or 3.5. In some embodiments of the present disclosure, the alkali is used in a form of an aqueous alkali solution, and a concentration of the aqueous alkali solution is in a range of 20 wt% to 40 wt%, and is 20 wt%, 25 wt%, 30 wt%, 35 wt% or 40 wt% in a particular embodiment. In some embodiments of the present disclosure, the aqueous alkali solution is added in a dropwise manner, and during dropwise addition, a temperature of a system is in a range of 15°C to 25°C, and is 15°C, 20°C or 25°C in a particular embodiment.

In the present disclosure, the water-immiscible solvent and the ice water are used as a hydrolysis system for phosphorus oxychloride and the pH is adjusted to 3 to 3.5, such that a by-product from phosphorus oxychloride during the chlorination reaction hydrolyzes to phosphoric acid. Excess phosphorus oxychloride hydrolyzes to phosphoric acid and hydrochloric acid. The hydrochloric acid is then neutralized to sodium chloride, and the phosphoric acid is retained. Furthermore, such pH range allows for the complete separation of the chlorides from water by extraction using the water-immiscible solvent.

In some embodiments of the present disclosure, the chlorides include 3,6-dichloropyridazine or 2-chloroquinoxaline.

In some embodiments of the present disclosure, after obtaining the acidic aqueous phase, the process further includes: decolorizing the acidic aqueous phase by using a fourth activated carbon to obtain a decolorized aqueous acid solution; and then recycling the decolorized aqueous acid solution back to the step of adjusting the pH to 5.5 to 6.0 (i.e., to adjust a pH of a resulting system after decolorization by using the first activated carbon). In the present disclosure, the acidic aqueous phase is recycled back to a post-treatment step after the condensation reaction to achieve the full utilization of the phosphorus-containing spent acid produced during the preparation of the sulfonamide compound.

In some embodiments of the present disclosure, decolorizing the acidic aqueous phase by using the fourth activated carbon is conducted at a temperature of 20°C to 30°C, and is 20°C, 25°C or 30°C in a particular embodiment; and decolorizing the acidic aqueous phase by using the fourth activated carbon is conducted for 1 h to 2 h, and is 60 min, 70 min, 80 min, 90 min, 100 min, 110 min or 120 min in a particular embodiment. In some embodiments of the present disclosure, a mass ratio of the hydroxyl compound to the activated carbon is in a range of 1 : 0.02-0.04, and is 1 : 0.02, 1 : 0.03 or 1 : 0.04 in a particular embodiment.

In some embodiments of the present disclosure, after the chloride is obtained, the chloride, the sulfonamide, the organic solvent, and the alkaline reagent are mixed and subjected to a condensation reaction to obtain the crude product solution of the sulfonamide compound.

In some embodiments of the present disclosure, a molar ratio of the sulfonamide to the chloride is in a range of 1 : 0.95-1, and is 1 : 0.95, 1 : 0.96, 1 : 0.97, 1 : 0.98, 1 : 0.99, or 1 : 1 in a particular embodiment.

In some embodiments of the present disclosure, the alkaline reagent includes sodium carbonate and/or sodium bicarbonate. In some embodiments of the present disclosure, a molar ratio of the sulfonamide to the alkaline reagent is in a range of 1 : 1-1.45, and is 1 : 1, 1 : 1.1, 1 : 1.2, 1 : 1.3, 1 : 1.4, or 1 : 1.45 in a particular embodiment.

In some embodiments of the present disclosure, the organic solvent includes o-dichlorobenzene and/or dimethylformamide (DMF). In some embodiments of the present disclosure, a ratio of a mass of the chloride to a volume of the organic solvent is in a range of 1 g: 4.5-5.5 mL, and is 1 g : 4.5 mL, 1 g : 5 mL, or 1 g : 5.5 mL in a particular embodiment.

In some embodiments of the present disclosure, the condensation reaction is conducted at a temperature of 145°C to 170°C, and is 145°C, 150°C, 155°C, 160°C, 165°C, or 170°C in a particular embodiment; and the condensation reaction is conducted for 3.5 h to 4 h, and is 210 min, 220 min, 230 min or 240 min in a particular embodiment.

In the present disclosure, after the crude product solution of the sulfonamide compound is obtained, the solvent in the crude product solution of the sulfonamide compound is removed and then the water is added, or the water is added to the crude product solution of the sulfonamide compound and a resulting mixture is subjected to layering to obtain the condensed aqueous phase, and the pH of the condensed aqueous phase is adjusted to 5.5 to 6.0, and a resulting system is subjected to solid-liquid separation to obtain the first solid component and the first mother liquor, respectively; and the first solid component is dried to obtain the sulfonamide compound; where the crude product solution of the sulfonamide compound is obtained by subjecting the phosphorus oxychloride and the hydroxyl compound to a chlorination reaction, and subjecting a resulting reaction product to acidification at a pH of 3 to 3.5 and then subjecting a resulting acidification product to a condensation reaction with sulfonamide; and the acid for adjusting the pH to 5.5 to 6.0 includes the acid obtained by the acidification.

In some embodiments of the present disclosure, the water is hot water, and a temperature of the hot water is in a range of 80°C to 100°C, and is 80°C, 85°C, 90°C, 95°C, or 100°C in a particular embodiment.

In the present disclosure, there is no limitation on the method for removing the solvent in the crude product solution of the sulfonamide compound, and any solvent removal method well known to those skilled in the art can be used, specifically such as evaporation.

In some embodiments of the present disclosure, the condensed aqueous phase is first subjected to decolorization by using a first activated carbon and then adjusted to a pH of 5.5 to 6.0. In some embodiments of the present disclosure, the decolorization by using the first activated carbon is conducted at a temperature of 85°C to 95°C, and is 85°C, 90°C or 95°C in a particular embodiment; and the decolorization by using the first activated carbon is conducted for 1 h to 2 h, and is 60 min, 70 min, 80 min, 90 min, 100 min, 110 min or 120 min in a particular embodiment. In some embodiments of the present disclosure, a mass ratio of the chloride to the activated carbon is in a range of 1 : 0.02-0.04, and is 1 : 0.02, 1 : 0.03 or 1 : 0.04 in a particular embodiment.

In some embodiments of the present disclosure, the pH is 5.5, 5.6, 5.7, 5.8, 5.9 or 6.0. In some embodiments of the present disclosure, the acid for adjusting the pH to 5.5 to 6.0 is the decolorized aqueous acid solution.

In some embodiments of the present disclosure, the solid-liquid separation is conducted while the resulting system is still hot. In the present disclosure, there are no particular limitations on the solid-liquid separation, and any solid-liquid separation method well known to those skilled in the art can be used, specifically such as filtration, suction filtration, or centrifugation.

In some embodiments of the present disclosure, the drying is conducted at a temperature of 95°C to 105°C, and is 95°C, 98°C, 100°C, 102°C, or 105°C in a particular embodiment. In the present disclosure, there is no limitation on the time of the drying, as long as the product can be dried to a constant weight.

In the present disclosure, the phosphorus oxychloride and the hydroxyl compound undergo a chlorination reaction, followed by acidification at a pH of 3 to 3.5 to produce the chloride, hydrochloric acid, and phosphoric acid, and then a condensation reaction with sulfonamide to produce the sulfonamide compound and sodium carbonate. In the present disclosure, the method reduces acid and alkali consumption in the production of the sulfonamide compound and in the treatment of phosphorus-containing wastewater by means of the neutralization between the hydrochloric acid and phosphoric acid (acidic aqueous phases) produced during the preparation of the sulfonamide compound and sodium carbonate produced during the condensation reaction. The pH of the condensed aqueous phase is adjusted to 5.5 to 6.0 to produce sodium dihydrogen phosphate and disodium hydrogen phosphate.

In the present disclosure, after the first mother liquor is obtained, the pH of the first mother liquor is adjusted to 12 to 13 by using the sodium hydroxide, and the resulting system was cooled to 0°C to 10°C, and subjected to crystallization and solid-liquid separation in sequence to obtain a second solid component and a second mother liquor, respectively; and the second solid component is dried to obtain the trisodium phosphate.

In some embodiments of the present disclosure, the first mother liquor is first subjected to decolorization by using a second activated carbon and solid-liquid separation in sequence, and a resulting liquid component is then adjusted to a pH of 12 to 13 by using sodium hydroxide. In some embodiments of the present disclosure, the decolorization by using the second activated carbon is conducted at a temperature of 20°C to 30°C, and is 20°C, 25°C or 30°C in a particular embodiment; the decolorization by using the second activated carbon is conducted for 1 h to 2 h, and is 60 min, 70 min, 80 min, 90 min, 100 min, 110 min or 120 min in a particular embodiment. and a mass ratio of the chloride to the activated carbon is in a range of 1 : 0.02-0.04, and is 1 : 0.02, 1 : 0.03 or 1 : 0.04 in a particular embodiment.

In the present disclosure, there are no particular limitations on the solid-liquid separation, and any solid-liquid separation method well known to those skilled in the art can be used, specifically such as filtration, suction filtration, or centrifugation.

In some embodiments of the present disclosure, the pH is 12, 12.2, 12.4, 12.5, 12.6, 12.8 or 13. In the present disclosure, sodium hydroxide is used to adjust the pH to 12 to 13, so that the sodium dihydrogen phosphate and the disodium hydrogen phosphate are completely converted to the trisodium phosphate, the resulting trisodium phosphate is present in a form of trisodium phosphate dodecahydrate, and the trisodium phosphate dodecahydrate may be dried to obtain trisodium phosphate hexahydrate and/or anhydrous trisodium phosphate.

In some embodiments of the present disclosure, the method further includes, before cooling to 0°C to 10°C, decolorizing a system with pH being adjusted to 12 to 13 by using a third activated carbon. In some embodiments of the present disclosure, the decolorizing by using the third activated carbon is conducted at a temperature of 85°C to 95°C, and is 85°C, 90°C or 95°C in a particular embodiment; the decolorization by using the third activated carbon is conducted for 1 h to 2 h, and is 60 min, 70 min, 80 min, 90 min, 100 min, 110 min or 120 min in a particular embodiment; and a mass ratio of the chloride to an activated carbon is in a range of 1 : 0.02-0.04, and is 1 : 0.02, 1 : 0.03 or 1 : 0.04 in a particular embodiment.

In some embodiments of the present disclosure, the system is cooled to a temperature of 0°C, 2°C, 4°C, 5°C, 6°C, 8°C or 10°C; and the crystallization is conducted for 1 h to 2 h, and is 60 min, 70 min, 80 min, 90 min, 100 min, 110 min or 120 min in a particular embodiment. In the present disclosure, the trisodium phosphate is separated by utilizing the significant difference in solubility between the trisodium phosphate and sodium chloride at a low temperature of 0°C to 10°C.

In the present disclosure, the drying is conducted by air drying at room temperature.

In the present disclosure, after the second mother liquor is obtained, the second mother liquor and the calcium chloride are mixed, and subjected to a precipitation reaction, and a resulting reaction product is subjected to solid-liquid separation to obtain a third solid component and a final mother liquor, respectively; and the third solid component is dried to obtain the calcium phosphate.

In some embodiments of the present disclosure, a molar ratio of the calcium chloride to the trisodium phosphate in the second mother liquor is in a range of 1.5-1.6 : 1, and is 1.51 : 1, 1.52 : 1, 1.53 : 1, 1.54 : 1, 1.55 : 1, 1.56 : 1, 1.57 : 1, 1.58 : 1, 1.59 : 1 or 1.6 in a particular embodiment; and the calcium chloride includes calcium chloride dihydrate. In the present disclosure, by controlling the ratio of the amount of trisodium phosphate in the second mother liquor to calcium chloride, the method enables the conversion of trisodium phosphate in the second mother liquor into calcium phosphate, which can be removed by solid-liquid separation, and thus the purpose of phosphorus removal from wastewater is achieved.

In the present disclosure, the precipitation reaction is conducted at room temperature for 1 h to 1.5 h, and is 60 min, 70 min, 80 min or 900 min in a particular embodiment; and the precipitation reaction is conducted under a stirring condition.

In the present disclosure, there are no particular limitations on the solid-liquid separation, and any solid-liquid separation method well known to those skilled in the art can be used, specifically such as filtration, suction filtration, or centrifugation.

In the present disclosure, calcium chloride and sodium phosphate are reacted to form a calcium phosphate precipitate, and thus phosphate ions are substantially removed from the second mother liquor via solid-liquid separation, and sodium chloride remains in the final mother liquor, thereby achieving the full recovery of inorganic salts from the phosphorus-containing wastewater and the goal of advanced phosphorus removal from the phosphorus-containing wastewater.

In the present disclosure, the drying is conducted at a temperature of 95°C to 105°C, and is 95°C, 98°C, 100°C, 102°C, or 105°C in a particular embodiment. In the present disclosure, there is no limitation on drying time, as long as the product can be dried to a constant weight.

In some embodiments of the present disclosure, the method further includes, after obtaining the final mother liquor, concentrating and drying the final mother liquor in sequence to obtain sodium chloride. In the present disclosure, there is no limitation on the concentration and drying, as long as the solvent in the final mother liquor can be removed.

The present disclosure provides a novel idea for treating phosphorus-containing wastewater and considers the treatment of phosphorus-containing wastewater in the process design stage, thereby offering a new alternative for treating the phosphorus-containing wastewater from other similar products. Furthermore, the method can reduce the production costs of enterprises, and in particular can reduce the cost of solid waste treatment.

To further illustrate the present disclosure, the method for treating the phosphorus-containing wastewater produced during the preparation of the sulfonamide compound provided by the present disclosure will be described in detail below in conjunction with examples, which cannot be construed as limiting the scope of the present disclosure, however.

### Example 1

S1 Chlorination reaction: 475 g of phosphorus oxychloride, and 170 g of maleic hydrazide were added to a reaction flask, a hydrogen chloride absorption apparatus was connected, and stirring was started. A resulting system was heated to 100°C and held at 100°C, and subjected to reaction for 2 h and then cooled to 20°C to obtain a chlorination reaction solution.

S2 Hydrolysis reaction: 850 mL of dichloromethane and 510 mL of ice water were added to a reaction flask, the chlorination reaction solution was added thereto dropwise at 15°C, then a 30 wt% aqueous sodium hydroxide solution was added thereto dropwise to adjust pH to 3. A resulting system was layered to obtain an organic phase and an acidic aqueous phase, respectively. The organic phase was evaporated to dryness to obtain 220 g of 3,6-dichloropyridazine. 5.1 g of activated carbon was added to the acidic aqueous phase, and decolorization was conducted at 30°C for 1 h, obtaining a decolorized aqueous acid solution.

S3 Condensation reaction: 1100 mL of o-dichlorobenzene, 220 g of the 3,6-dichloropyridazine, 268 g of sulfonamide, and 224 g of sodium carbonate were added to a reaction flask, stirring was started. A resulting system was heated to 165°C and held at 165°C for 3.5 h. 1100 mL of hot water at 80°C was added thereto, and layering was conducted. A resulting aqueous phase was decolorized at 95°C for 1 h by adding 6.6 g of activated carbon and then neutralized by using the decolorized aqueous acid solution obtained in S2 at 95°C to pH to 5.5. A resulting system was filtered while the resulting system was still hot to obtain a first solid component and a first mother liquor, respectively. The first solid component was dried to obtain sulfachloropyridazine.

The first mother liquor was decolorized at 20°C for 2 h by adding 6.6 g of activated carbon. A resulting system was filtered to remove the activated carbon and water insoluble substances. Sodium hydroxide was added thereto to adjust pH to 13, then 6.6 g of activated carbon was added thereto, and decolorization was conducted at 80°C for 1 h. A resulting system was cooled to 0°C and held at 0°C for 1 h and then filtered to obtain a second solid component and a second mother liquor, respectively. The second solid component was air dried at room temperature to obtain 1116 g of trisodium phosphate dodecahydrate, with a purity of 98.8%, equivalent to 1103 g of pure trisodium phosphate dodecahydrate (theoretical 1178 g), and the second mother liquor was estimated to contain about 75 g of the trisodium phosphate dodecahydrate.

45 g of calcium chloride dihydrate was added to the second mother liquor. A resulting system was stirred for 1 h and filtered to obtain a third solid component and a final mother liquor respectively. The third solid component was dried to obtain calcium phosphate. A phosphorus content in the final mother liquor was < 10 mg/L.

### Example 2

S1 Chlorination reaction: 420 g of phosphorus oxychloride, and 200 g of 2-hydroxyquinoxaline were added to a reaction flask, a hydrogen chloride absorption apparatus was connected, and stirring was started. A resulting mixture was heated to 105°C and held at 105°C, and subjected to reaction for 1.5 h. The phosphorus oxychloride was then recovered under reduced pressure, and a resulting system was cooled to 30°C to obtain a chlorination reaction solution.

S2 Hydrolysis reaction: 1000 mL of dichloromethane and 600 mL of ice water were added to a reaction flask, the chlorination reaction solution was added thereto dropwise at 25°C, then a 30 wt% aqueous sodium hydroxide solution was added thereto dropwise to adjust pH to 3.5. A resulting system was layered to obtain an organic phase and an acidic aqueous phase, respectively. The organic phase was evaporated to dryness to obtain 220 g of 2-chloroquinoxaline. 6 g of activated carbon was added to the acidic aqueous phase, and decolorization was conducted at 20°C for 2 h, obtaining a decolorized aqueous acid solution.

S3 Condensation reaction: 1100 mL of anhydrous DMF, 220 g of the 2-chloroquinoxaline, 230 g of sulfonamide, and 146 g of sodium carbonate were added to a reaction flask, stirring was started. A resulting system was heated to 145°C to 150°C to evaporate an aqueous DMF at atmospheric pressure and held at 145°C to 150°C for 4 h. 600 mL of hot water at 100°C was added, and layering was conducted. A resulting aqueous phase was decolorized at 85°C for 2 h by adding 6.6 g of activated carbon and then neutralized by using the decolorized aqueous acid solution obtained in S2 at 85°C to pH to 6.0. A resulting system was filtered while the resulting system was still hot to obtain a first solid component and a first mother liquor, respectively. The first solid component was dried to obtain sulfaquinoxaline.

The first mother liquor was decolorized at 30°C for 1 h by adding 6.6 g of activated carbon. A resulting system was filtered to remove the activated carbon and water insoluble substances. Sodium hydroxide was added thereto to adjust pH to 12, then 6.6 g of activated carbon was added thereto, and decolorization was conducted at 70°C for 2 h. A resulting system was cooled to 10°C and held at 10°C for 1 h and then filtered to obtain a second solid component and a second mother liquor, respectively. The second solid component was air dried at room temperature to obtain 489 g of trisodium phosphate dodecahydrate, with a purity of 98.2%, equivalent to 489 g of pure trisodium phosphate dodecahydrate (theoretical 521 g). The second mother liquor was estimated to contain about 32 g of trisodium phosphate dodecahydrate.

19.5 g of calcium chloride dihydrate was added to the second mother liquor. A resulting system was stirred for 1 h and filtered to obtain a third solid component and a final mother liquor, respectively. The third solid component was dried to obtain calcium phosphate.

The phosphorus content in the final mother liquor was < 10 mg/L.

The descriptions above are merely the preferred embodiments of the present disclosure. It should be noted that several improvements and modifications may also be made by those of ordinary skill in the art without departing from the principle of the present disclosure, and these improvements and modifications shall also be considered within the scope of the present disclosure.

What is claimed is:

1. A method for treating phosphorus-containing wastewater produced during preparation of a sulfonamide compound, comprising:
removing a solvent in a crude product solution of the sulfonamide compound and then adding water, or adding the water to the crude product solution of the sulfonamide compound and layering, to obtain a condensed aqueous phase, adjusting a pH of the condensed aqueous phase to 5.5 to 6.0 and conducting a first solid-liquid separation to obtain a first solid component and a first mother liquor, respectively; and drying the first solid component to obtain the sulfonamide compound; wherein the crude product solution of the sulfonamide compound is prepared by a process comprising:
subjecting phosphorus oxychloride and a hydroxyl compound to a chlorination reaction to obtain a chlorination reaction solution;
mixing the chlorination reaction solution obtained after the chlorination reaction, a water-immiscible solvent, and ice water, adjusting a pH of a resulting mixture to 3 to 3.5, and layering to obtain an organic phase and an acidic aqueous phase, respectively; evaporating the organic phase to dryness to obtain a chloride; and recycling the acidic aqueous phase back to a step of adjusting the pH of the condensed aqueous phase to 5.5 to 6.0; and
mixing the chloride, sulfonamide, an organic solvent, and an alkaline reagent, and conducting a condensation reaction to obtain the crude product solution of the sulfonamide compound,
adjusting a pH of the first mother liquor to 12 to 13 by using sodium hydroxide, cooling a resulting material to 0° C to 10° C, and conducting crystallization and a second solid-liquid separation in sequence to obtain a second solid component and a second mother liquor, respectively; and drying the second solid component to obtain trisodium phosphate; and
mixing the second mother liquor and calcium chloride and conducting a precipitation reaction, and subjecting a resulting reaction material to a third solid-liquid separation to obtain a third solid component and a final mother liquor, respectively; and drying the third solid component to obtain calcium phosphate.

2. The method of claim 1, wherein the method further comprises:
decolorizing the condensed aqueous phase by using a first activated carbon before adjusting the pH of the condensed aqueous phase to 5.5 to 6.0; and
decolorizing the first mother liquor by using a second activated carbon and then conducting a fourth solid-liquid separation before adjusting the pH of the first mother liquor to 12 to 13 by using the sodium hydroxide.

3. The method of claim 1, wherein the method further comprises, before cooling to 0° C to 10° C, decolorizing the resulting material by using a third activated carbon; and
the crystallization is conducted for 1 hour to 2 hours.

4. The method of claim 1, wherein a molar ratio of the calcium chloride to trisodium phosphate in the second mother liquor is in a range of 1.5-1.6 : 1.

5. The method of claim 1, wherein the hydroxyl compound is selected from a group consisting of maleic hydrazide and 2-hydroxyquinoxaline;
a molar ratio of the hydroxyl compound to the phosphorus oxychloride is in a range of 1 : 2-2.2; and
the chlorination reaction is conducted at a temperature of 100° C to 105° C for 1.5 hours to 2 hours.

6. The method of claim 1, wherein the water-immiscible solvent comprises at least one selected from the group consisting of dichloromethane, chloroform, dichloroethane, and toluene; and
an alkali for adjusting the pH of the resulting mixture to 3 to 3.5 comprises at least one selected from the group consisting of sodium hydroxide, sodium carbonate, and sodium bicarbonate.

7. The method of claim 1, wherein a molar ratio of the sulfonamide to the chloride is in a range of 1 : 0.95-1;
the alkaline reagent comprises at least one selected from the group consisting of sodium carbonate and sodium bicarbonate, and a molar ratio of the sulfonamide to the alkaline reagent is in a range of 1: 1-1.45; and
the organic solvent comprises at least one selected from the group consisting of o-dichlorobenzene and dimethylformamide.

8. The method of claim 1, wherein the condensation reaction is conducted at a temperature of 145° C to 170° C for 3.5 hours to 4 hours.

9. The method of claim 1, wherein the process further comprises decolorizing the acidic aqueous phase by using a fourth activated carbon before use.

10. The method of claim 7, wherein the condensation reaction is conducted at a temperature of 145° C to 170° C for 3.5 hours to 4 hours.
